(19) United States
(12) Patent Application Publication
(10) Pub. No.: US 2014/0005794 A1
(43) Pub. Date: Jan. 2, 2014

(54) GRANULATE MIXTURE COMPRISING TWO DIFFERENT GRANULATES FOR ARTIFICIAL CALLUS DISTRACTION

(71) Applicant: Domonkos Horvath, Jestetten (DE)

(72) Inventor: Domonkos Horvath, Jestetten (DE)

(73) Assignee: CELGEN AG, Zug (CH)

(21) Appl. No.: 14/005,422

(22) PCT Filed: Mar. 10, 2012
(86) PCT No.: PCT/EP2012/001081

§ 371 Date: Sep. 16, 2013

(30) Foreign Application Priority Data
Mar. 14, 2011 (DE) 10 2011 014 789.6

(51) Int. Cl. A61L 27/40; A61F 2/28

(52) U.S. Cl. CPC A61L 27/40; A61F 2/28
USPC 623/23.5; 523/116

(57) ABSTRACT

The present invention relates to a granulated material mixture for regenerating bone, in particular by way of three-dimensional callus distraction, and to uses of said granulated material mixture. The granulated material mixture comprises rigid and deformable granulated materials.

The present invention relates to a granulated material mixture for regenerating bone, in particular by way of three-dimensional distraction, to methods for three-dimensional callus distraction, and to uses of said granulated material mixture.

Bone losses are currently generally filled with bone substitute material or with autogenous or allogeneic bone.

Examples of bone substitute materials include inorganic materials such as calcium phosphates, hydroxylapatite or bioglass. These will be replaced with bone after slow resorption. However, this procedure can only be used for minor defects because there is otherwise a risk of infection due to insufficient vascularization. Such bone substitute materials do not emit biomechanical pulses and therefore do not trigger active regeneration. In addition, synthetically produced bone substitute materials are available, which are made of organic materials, such as polyesters, polyamino acids, polyanhydrides, polyorthoesters, polyphosphazenes, polylactides, or polyglycolides, or are made of allogeneic organic materials, which are of bovine origin, for example. However, bone matter losses can also be bridged using microvascular connected autogenous or allogeneic vascularized transplants.

From a biological view, the best substitute material for bone is an autologous spongiosa transplant. However, such transplants are only available to a limited extent and exhibit a high resorption rate after transplantation.

The materials and techniques employed in the prior art frequently provide inadequate bone quality, so that implants, for example, are not rigidly anchored in the beds thereof. Additionally, the bone substitute is frequently not sufficiently vascularized, and as a result the risk of infection is increased. Methods according to the prior art often also employ growth factors, which significantly increase the costs of the procedures.

Bone substitute materials are frequently used in the form of a granulated material, in particular in the mouth and jaw region. Such a granulated material is described in WO 20061010507 A2, for example. Examples of granulated materials known on the market include Bio-Oss® from Geistlich Pharma AG, BONIT Matrix® from DOT GmbH, and cyclOS® and Ceros® from Mathys AG.

Instead of using a bone substitute, missing bone matter can also be partially filled in by way of bone regeneration. Segmental osseous discontinuity on long bones can thus be treated by way of distraction osteogenesis.

Callus distraction has been known for more than one hundred years. The most important biological stimulus for osteogenesis is mechanical stress. Piezoelectrical forces are released in the process, which activate osteoblasts and osteoclasts. Distraction osteogenesis induces new bone formation by triggering biological growth stimuli by slowly separating bone segments. This method achieves direct formation of woven bone by way of distraction. Defined tensile stress during bone generation is essential. If such defined tensile stress is applied to bone fragments, the mesenchymal tissues in the gap and on the adjoining fragment ends show osteogenetic capacity. If sufficient vascular potency exists, progressive distraction results in metaplasia of the organized hematoma, also referred to as a blood clot, in a zone of longitudinally arranged, fibrous tissue, which under optimal external and internal conditions can convert directly into woven bone. However, an complicating factor is that the bone tissue is subject to highly complex control during regeneration.

WO 01/91663 describes two-dimensionally oriented bone distraction using an artificial interface. Such distraction methods from the prior art frequently only allow vertical regeneration, for example in the jaw region.

Thus, bone regeneration by way of distraction cannot be used for every type of bone defect. In addition, the devices used for distraction are complex, and distraction procedures take a comparatively long time.

DE 10 2006 047 248 A1 describes a three-dimensional framework, which transmits pulses directly to osteoblasts, so as to activate them, by way of changes in volume.

The technical problem underlying the present invention is that of providing a device that makes it possible to carry out bone regeneration methods that overcome the drawbacks of the prior art. The technical object underlying the invention is also to provide devices that improve on the known devices for bone regeneration, in particular in a simple manner.

The technical object underlying the invention is also to provide devices, uses thereof, and methods that allow simple and cost-effective bone regeneration.

The technical problem underlying the present invention is also that of providing devices, uses thereof, and methods that make it possible to regenerate bone and provide improved quality and sufficient vascularization.

The present invention solves the underlying technical problem in particular by providing devices, methods and uses according to the claims.

The devices according to the invention are in particular granulated material mixtures according to the invention.

The present invention solves the underlying technical problem in particular by providing a granulated material mixture that is suitable for regenerating a bone and comprises at least one deformable particle and at least one non-deformable particle.

The present invention solves the underlying technical problem in particular also by providing a granulated material mixture that is suitable for regenerating a bone and comprises at least one expandable particle and at least one non-expandable particle.

According to the invention, the granulated material mixture preferably comprises a plurality of the deformable particles and a plurality of the non-deformable particles.

According to the invention, the granulated material mixture preferably comprises a plurality of the expandable particles and a plurality of the non-expandable particles.

According to the invention, in one alternative embodiment it may be provided that the granulated material mixture consists of at least one deformable particle and at least one non-deformable particle.

The granulated material mixture according to the invention can advantageously be used in methods, preferably in methods according to the invention, for bone regeneration, and more particularly for three-dimensional callus distraction.

The present teaching includes in particular granulated material mixtures and methods for bone regeneration, wherein preferably bones in the jaw region and/or in the periodontal region are to be regenerated.

In the present invention, the term ‘bone regeneration’ is understood, in particular, to also mean the regeneration of bone defects, for example after cystectomy, tumor surgery or trauma surgery or the like, regardless of the topography, and/or, in particular, also the regeneration of smaller bone defects caused by periodontitis, for example.

However, bone outside the jaw region and/or outside the periodontal region may also be regenerated.

According to the invention, preferably any mixing ratio of the deformable particles to the non-deformable particles may be provided in the granulated material mixture, as needed.

According to the invention, preferably any mixing ratio of the expandable particles to the non-expanded particles may be provided in the granulated material mixture, as needed.

According to the invention, the mixing ratio of the deformable particles to the non-deformable particles in the granulated material mixture, relative to the number of particles, preferably ranges from 1:999 to 999:1. According to the invention, the mixing ratio of the deformable particles to the non-deformable particles in the granulated material mixture, relative to the number of particles, preferably ranges from 1:99 to 99:1.

For example, it may be provided that the mixing ratio of the deformable particles to the non-deformable particles in the granulated material mixture, relative to the number of particles, ranges from 1:9 to 9:1.

According to the invention, the mixing ratio of the expandable particles to the non-expandable particles in the granulated material mixture, relative to the number of particles, preferably ranges from 1:999 to 999:1. According to the invention, the mixing ratio of the expandable particles to the non-expandable particles in the granulated material mixture, relative to the number of particles, preferably ranges from 1:99 to 99:1.

For example, it may be provided that the mixing ratio of the expandable particles to the non-expandable particles in the granulated material mixture, relative to the number of particles, ranges from 1:9 to 9:1.

For example, according to an alternative embodiment, the number of non-deformable particles present in the granulated material mixture may be greater than that of the deformable, and in particular expandable, particles.

According to the invention, the at least one deformable particle preferably comprises a swelling agent. For example, the at least one deformable particle may consist of a swelling agent.

According to the invention, the at least one expandable particle preferably comprises a swelling agent. For example, the at least one expandable particle may consist of a swelling agent.

In one alternative according to the invention, the swelling agent may be solid. In one alternative according to the invention, the swelling agent may be semi-solid. In one alternative according to the invention, the swelling agent may be present as a foam. In one alternative according to the invention, the swelling agent may be present as a powder, in particular if the swelling agent is surrounded by a covering or casing. In one alternative according to the invention, the swelling agent may be present in liquid form, in particular if the swelling agent is surrounded by a casing.

In one alternative according to the invention, the expandable particle may be solid. In one alternative according to the invention, the expandable particle may be semi-solid. In one alternative according to the invention, the expandable particle may be present as a foam. The expandable particle may be present, in particular, in solid, semi-solid or foam form if the expandable particle consists of a swelling agent.

According to the invention, the swelling agent is preferably biocompatible. According to the invention, the swelling agent is preferably biodegradable.

For example, it may be provided that the swelling agent is not biogenic, and more particularly that the swelling agent does not comprise any collagen or is collagen-free. However, it may also be provided that the swelling agent is biogenic.

For example, it may also be provided that the swelling agent of the at least one deformable, and in particular expandable, particle is enclosed by a biodegradable covering. The covering can be formed of one or more biodegradable materials. The undegraded, which is to say intact, covering prevents contact of the swelling agent with a liquid. After the covering has partially or completely degraded, the swelling agent can come in contact with a liquid.

By selecting the thickness of the covering, a person skilled in the art may determine the time frame over which the coating is dissolved. As a result, it is possible to predetermine the time at which the distraction starts. For example, the thicker the biodegradable covering is designed, the later the deformation will begin, and more particularly the expansion of the covered granulated material, and thus the onset of the distraction pulse.

The thickness of the covering can range from the thickness of a molecular film to 5 mm. According to the invention, the thickness of the covering is preferably at least that of a molecular film. According to the invention, the thickness of the covering preferably does not exceed 5 mm, in particular 2 mm, and more particularly 1 mm. In one embodiment according to the invention, the thickness of the covering is between 0.1 μm and 1 mm. In one embodiment according to the invention, the thickness of the covering is at least 10 μm and no more than 100 μm.

In one embodiment according to the invention, the covering can have a resorption time of at least one day, for example. In one embodiment according to the invention, the covering can have a resorption time of at least five days, for example. In one embodiment according to the invention, the covering can have a resorption time of approximately one week, for example. In one embodiment according to the invention, the covering can have a resorption time of 4 days to 10 days, for example. In one embodiment according to the invention, the covering can have a resorption time of 6 days to 8 days, for example. In one embodiment according to the invention, the covering can have a resorption time of no more than 10 weeks, and more particularly of no more than 3 weeks, for example. In one embodiment according to the invention, the covering can have a resorption time of at least one day and no more than ten weeks, for example. In one embodiment according to the invention, the covering can have a resorption time of at least one day and no more than one week, for example. In one embodiment according to the invention, the covering can have a resorption time of at least one week and no more than ten weeks, for example.

In one embodiment according to the invention, the covering can consist of gelatin or comprise gelatin, for example. In one embodiment according to the invention, the covering can consist of substances that are gelatin-like or that have the properties of gelatin, or comprise the same, for example. A person skilled in the art will know how to distinguish such substances from galenics.

The use of gelatin or gelatin-like substances has the advantage that the breakdown of gelatin does not lower the pH value of the environment because no acid degradation products are created.

In one embodiment according to the invention, the covering can consist of at least one gelatin film, for example.

However, it may also be provided, for example, that the swelling agent of the at least one deformable, and in particular expandable, particle is enclosed by a biodegradable casing. According to the invention, the swelling agent is preferably located inside the casing, which is to say is surrounded by the casing. According to the invention, the casing thus preferably forms a cavity in which the swelling agent is located. According to the invention, preferably a portion of the cavity, and more particularly the entire cavity, that is formed by the casing is filled with the swelling agent. According to the invention, preferably the entire cavity that is formed by the casing is filled with the swelling agent. The cavity is delimited by the casing, even if the casing has openings, such as pores.

According to the invention, the casing preferably reacts to the change in volume of the swelling agent by undergoing expansion, deformation and/or shrinkage. According to the invention, the casing preferably reacts to the change in volume of the swelling agent by undergoing expansion. According to the invention, the casing preferably reacts to the change in volume of the swelling agent by undergoing deformation. According to the invention, the casing preferably reacts to the change in volume of the swelling agent by undergoing expansion and deformation.

According to the invention, the casing preferably comprises a material selected from the group consisting of polyglycolic acid, polylactic acid, poly(ε-caprolactone), poly(β-hydroxybutyrate), poly(p-dioxanone), a polyanhydride, or a mixture thereof, for example a mixture of polylactic acid and polyglycolic acid. According to the invention, the casing preferably comprises polylactic acid. According to the invention, the casing preferably comprises poly(ε-caprolactone). According to the invention, the casing preferably comprises a carbolactone.

According to the invention, the material of the casing preferably comprises copolymers, in particular made of at least two of the materials mentioned above. According to the invention, the material of the casing preferably comprises polymer mixtures.

According to the invention, the casing preferably consists of a material selected from the group consisting of polyglycolic acid, polylactic acid, poly(ε-caprolactone), poly(β-hydroxybutyrate), poly(p-dioxanone), a polyanhydride, or a mixture thereof. According to the invention, the material of the casing preferably consists of copolymers made of at least two of the materials mentioned above.

According to the invention, the casing preferably consists of polylactic acid. A casing that comprises polylactic acid, or consists of polylactic acid, has the advantage that that the polylactic acid degrades into short-chain metabolites. Moreover, polylactic acid imparts a certain degree of hardness to the casing.

According to the invention, the casing preferably consists of poly(ε-caprolactone). A casing that comprises poly(ε-caprolactone), or consists of poly(ε-caprolactone), has the advantage that poly(ε-caprolactone) is particularly biocompatible. It is also possible to form long chains of poly(ε-caprolactone). During decomposition, few or no free acids are formed from poly(e-caprolactone).

According to the invention, the casing preferably consists of carbolactone.

According to the invention, the casing preferably consists of at least one polymer, or comprises the same, and preferably spatially cross-linked polymers.

According to the invention, the material of the casing preferably consists of at least one fiber composite, or comprises the same. According to the invention, the material of the casing preferably consists of fibers of a fiber composite, or comprises the same.

In one embodiment according to the invention, the casing consists of gelatin or gelatin-like substances, or comprises the same.

According to the invention, the casing preferably has at least one cell-adhesive property, which is to say it is able to bind cells, in particular osteoblasts, fibroblasts and/or endothelial cells, and preferably is able to bind these specifically and selectively. According to the invention, the cell-adhesive property of the casing is preferably determined by the surface properties thereof.

According to the invention, the outside of the casing is preferably coated with cells, in particular endothelial cells and/or osteoblasts and/or fibroblasts, before the casing is introduced into a defect region of a bone.

According to the invention, the material of the casing is preferably smooth. According to the invention, the coating of the casing is preferably smooth. According to the invention, the material of the casing is preferably rough. According to the invention, the coating of the casing is preferably rough. A preferred rough surface according to the invention provides a larger surface for binding the osteoblasts.

According to the invention, the casing is preferably coated with hydroxylapatite. A preferred coating with hydroxylapatite according to the invention allows proteins to be adsorbed, which promotes binding.

According to the invention, the casing is preferably coated with a hydrogel. According to the invention, the hydrogel layer is preferably thin.

According to the invention, the casing is preferably coated with at least one protein. According to the invention, the at least one protein preferably comprises the amino acid sequence Arg-Gly-Asp, which is to say RGD. According to the invention, the casing is preferably coated with at least one peptide. According to the invention, the at least one peptide is preferably a peptide that initiates the cell adhesion. According to the invention, the at least one peptide is preferably an RGD peptide. According to the invention, the at least one peptide is preferably synthetically produced. According to the invention, the at least one peptide preferably comprises the amino acid sequence Arg-Gly-Asp, which is to say RGD. According to the invention, the at least one peptide preferably consists of the amino acid sequence Arg-Gly-Asp, which is to say RGD.

According to the invention, the casing is preferably coated with star-shaped polyethylene glycol polymers (star PEGs).

According to the invention, the at least one protein is preferably bound to the polyethylene glycol polymer coating, and particularly preferably it is covalently bound thereto. According to the invention, the at least one peptide is preferably bound to the polyethylene glycol polymer coating, and particularly preferably it is covalently bound thereto.

The adhesion of osteoblasts is a receptor-induced contact between the molecules of the extracellular matrix and the actin filaments of the cytoskeleton. This region is also referred to as the focal contact zone. Both molecules that assure binding and molecules that are responsible for signal transduction are present in the focal contacts. Formation of the focal adhesion is primarily caused by integrins. Integrins differ from other cell surface receptors in the bioaffinity thereof. Adhesion proteins in the form of an ultrathin coating on the casing facilitate the adhesive binding of osteoblasts to the device according to the invention. Fibronectin is an extracellular adhesion protein comprising several specific binding sites for receptors and is therefore used to bind the osteoblasts to the extracellular matrix. Fibronectin is a large glycoprotein and, being a dimer, is composed of two nearly identical subunits. Fibronectin is composed of some 90 amino acids. The cell-binding site of fibronectin was identified as the tripeptide sequence Arg-Gly-Asp (RGD).

According to the invention, the surface of the casing is preferably chemically modified. According to the invention, the surface of the casing is preferably chemically modified by reactive molecules or molecule groups. According to the invention, the molecules or molecule groups by which the surface of the casing is chemically modified can preferably react with anchor proteins of the extracellular matrix of cells. According to the invention, the surface of the casing is preferably hydrophilic. Hydrophilic surfaces allow for better adhesion of cells than hydrophobic surfaces.

According to the invention, the casing preferably has a thickness of at least 0.01 mm. According to the invention, the casing preferably has a thickness of no more than 1 mm. According to the invention, the casing preferably has a thickness of at least 0.05 mm and no more than 0.5 mm. According to the invention, the casing preferably has a thickness of approximately 0.1 mm.

According to the invention, the casing is preferably permeable to a liquid. According to the invention, the casing is preferably permeable to water. According to the invention, the casing is preferably porous. According to the invention, the casing preferably has pores that are permeable to water and to solids, such as proteins and sugars, having a mass of less than 100 kDa, and particularly preferably of less than 50 kDa. According to the invention, the casing preferably has pores that are not permeable to solids, such as proteins and sugars, having a mass of more than 50 kDa, particularly preferably of more than 100 kDa, and in particular of more than 150 kDa. According to the invention, the pores preferably have a size of no more than 2 μm, and particularly preferably of no more than 1 μm. According to the invention, the pores preferably have a size of no more than 0.5 μm, and particularly preferably of no more than 0.1 μm. According to the invention, the pores preferably have a size of at least 0.01 μm, and particularly preferably of at least 0.05 μm. According to the invention, the pores preferably have a size of at least 0.1 μm, and particularly preferably of at least 0.5 μm. According to the invention, the pores preferably have a size of 1 μm.

According to the invention, at least a portion of the casing preferably has the form of a bellows.

According to the invention, at least a portion of the casing preferably has the form of a corrugated tube.

According to the invention, the casing preferably has the form of a bellows.

According to the invention, the casing preferably has the form of a corrugated tube.

As with the equivalent portion of a bendable drinking straw, the portion of the casing formed as a bellows or corrugated tube can be pulled apart or pushed together.

The bellows or corrugated tube is preferably composed of at least one, particularly preferably at least two, and in particular a plurality of pleats.

According to the invention, the pleats of the bellows or corrugated tube preferably have a length of 0.5 mm to 2 mm, calculated from the inner circumference of the casing to the distal end of the pleats, which essentially form the outer circumference. According to the invention, the pleats of the bellows or corrugated tube preferably have a length of 1 mm.

According to the invention, the at least one portion of the casing formed as a bellows or corrugated tube is preferably pushed together in the starting state, which is to say prior to use of the particles. As a result of the change in volume, and notably the increase in volume, of the swelling agent, the preferred at least one portion of the casing formed as a bellows or corrugated tube is pushed apart.

According to the invention, the outer surface of the casing is preferably enlarged, in particular by the provision of contours. This enlargement not only increases the surface available for the cells, but also influences the organization of cell growth.

According to the invention, the outer surface of the casing is preferably enlarged by lamellae. In a preferred embodiment of the present invention, the lamellae are rod- or tube-like appendages. In a further particularly preferred embodiment, the lamellae are planar appendages, in particular wall-like, plate-like, leaf-like, fan-like, wing-like or other planar appendages. In a further preferred embodiment, the lamellae have enlarged surfaces, in particular by way of lamellae substructures, branches, protuberances or net-like structures.

According to the invention, the outer side of the casing preferably carries at least one lamella. According to the invention, the outer side of the casing preferably carries at least two lamellae. According to the invention, the outer side of the casing preferably carries a plurality of lamellae. According to the invention, the outer side of the casing preferably carries 2 to 20 lamellae.

According to the invention, the at least one lamella can preferably be part of the casing. According to the invention, the at least one lamella is preferably made of the same material as the casing.

According to the invention, the at least one lamella is preferably not part of the casing. According to the invention, the at least one lamella is preferably made of a different material than the casing.

According to the invention, the casing is preferably biocompatible. According to the invention, the casing is preferably biodegradable. According to the invention, the casing and/or the swelling agent are preferably biodegradable.

In the context of the present invention, “biodegradable” shall be understood to mean that the material can be degraded or resorbed by way of hydrolysis, polymer dissolution, enzymatic degradation and/or dissociation of the material constituents, preferably in an organism, for example a human or animal organism. According to the invention, the degradation products of the particles preferably have a relative molar mass of no more than 50,000 g/mol, and particularly preferably of no more than 40,000 g/mol. This allows them to be excreted in the normal way.

According to the invention, the biodegradable, deformable particles are preferably degraded in an organism within a resorption time of two years, particularly preferably within one year, in particular within one month, and most preferably within two weeks.

According to the invention, resorption preferably begins at 6 weeks after the particles have been introduced into an organism.

According to the invention, the resorption time of the biodegradable, deformable particles, in particular of the casing and/or of the swelling agent, is preferably at least four weeks, particularly preferably at least eight weeks, in particular 16 weeks, and most preferably at least 32 weeks. According to the invention, the resorption time of the biodegradable, deformable particles is preferably no more than 52 weeks, particularly preferably no more than 38 weeks, still more preferably no more than 16 weeks, and most preferably no more than eight weeks.

According to the invention, the biodegradable, deformable particles can preferably be biologically decomposed. According to the invention, the components of the device, notably the casing and the swelling agent, can preferably be biologically decomposed.

In one alternative embodiment according to the invention, the deformable, and in particular expandable, particles have no casing.

In one alternative embodiment according to the invention, the deformable, and in particular expandable, particles have no covering.

In one alternative embodiment according to the invention, the deformable, and in particular expandable, particles are porous. In one alternative embodiment according to the invention, the deformable, and in particular expandable, particles are not porous.

According to one embodiment, the swelling agent may be a hydrogel.

According to the invention, the hydrogel is preferably carboxymethylcellulose. According to the invention, the hydrogel preferably comprises carboxymethylcellulose. According to the invention, the hydrogel preferably consists of a polysaccharide. According to the invention, the hydrogel preferably comprises at least one polysaccharide. According to the invention, the hydrogel is preferably hyaluronic acid. According to the invention, the hydrogel preferably comprises hyaluronic acid. According to the invention, the swelling agent preferably comprises different components, in particular mixtures of the components disclosed herein, such as carboxymethylcellulose, polysaccharides and/or hyaluronic acid.

In one embodiment according to the invention, the hydrogel can be polyethylene glycol (PEG). In one embodiment according to the invention, the hydrogel can comprise polyethylene glycol (PEG). In one embodiment according to the invention, the hydrogel can be polyacrylamide. In one embodiment according to the invention, the hydrogel can comprise polyacrylamide.

In one embodiment, it may be provided in particular that the swelling agent of the at least one deformable, and in particular expandable, particle consists of a polysaccharide.

In one embodiment, it may be provided in particular that the swelling agent of the at least one deformable, and in particular expandable, particle consists of glucosamine.

In one embodiment, it may be provided in particular that the plurality of deformable, and in particular expandable, particles are made of the same material or different materials.

According to the invention, the deformation, and in particular the expansion, is preferably triggered by the swelling agent absorbing liquid, preferably a liquid comprising biomolecules and/or cells, particularly preferably blood.

According to the invention, the at least one deformable particle preferably has a particle size of 0.1 μm to 10 mm.

According to the invention, the at least one expandable particle preferably has a particle size of 0.1 μm to 10 mm.

In one alternative embodiment according to the invention, the at least one deformable particle has a particle size of 0.1 mm to 10 mm.

In one alternative embodiment according to the invention, the at least one expandable particle has a particle size of 0.1 mm to 10 mm.

In one alternative embodiment according to the invention, the at least one deformable particle has a particle size of at least 0.1 μm. In one alternative embodiment according to the invention, the at least one deformable particle has a particle size of at least 1 μm. In one alternative embodiment according to the invention, the at least one deformable particle has a particle size of at least 10 μm. In one alternative embodiment according to the invention, the at least one deformable particle has a particle size of at least 100 μm. In one alternative embodiment according to the invention, the at least one deformable particle has a particle size of at least 200 μm.

In one alternative embodiment according to the invention, the at least one deformable particle has a particle size of no more than 10 mm. In one alternative embodiment according to the invention, the at least one deformable particle has a particle size of no more than 5 mm. In one alternative embodiment according to the invention, the at least one deformable particle has a particle size of no more than 1 mm.

In one preferred embodiment, the particle size of the deformable and non-deformable particles is at least 0.0001 mm. In one preferred embodiment, the particle size of the deformable and non-deformable particles is at least 0.001 mm. In one preferred embodiment, the particle size of the deformable and non-deformable particles is at least 0.01 mm.

In one preferred embodiment, the deformable and/or the non-deformable particles are not present in powder form, but in granular form. In one preferred embodiment, the deformable and the non-deformable particles are not present in powder form, but in granular form.

In one embodiment according to the invention, the particle size is 0.2 mm to 5 mm. In one embodiment according to the invention, the particle size is 0.5 mm to 5 mm. In one embodiment according to the invention, the particle size is 0.6 mm to 5 mm. In one embodiment according to the invention, the particle size is 0.5 mm to 4 mm.

In one preferred embodiment, the particle size is at least 0.001 mm. In one preferred embodiment, the particle size is at least 0.01 mm.

In one embodiment according to the invention, the particle size is at least 0.0001 mm. In one embodiment according to the invention, the particle size is at least 0.1 mm. In one embodiment according to the invention, the particle size is at least 0.2 mm. In one embodiment according to the invention, the particle size is at least 0.3 mm. In one embodiment according to the invention, the particle size is at least 0.4 mm. In one embodiment according to the invention, the particle size is at least 0.5 mm. In one embodiment according to the invention, the particle size is at least 0.6 mm.

In one embodiment according to the invention, the particle size is no more than 10 mm. In one embodiment according to the invention, the particle size is no more than 5 mm. In one embodiment according to the invention, the particle size is no more than 4 mm. In one embodiment according to the invention, the particle size is no more than 2 mm. In one embodiment according to the invention, the particle size is no more than 1 mm.

The information regarding the particle sizes of the deformable, and in particular expandable, particles refers to the size of the particles in the starting state, which is to say in the undeformed and/or unexpanded state.

In one alternative embodiment, it may be provided in particular that the particles of the plurality of deformable, and in particular expandable, particles are present in a single particle size.

However, in one alternative embodiment, it may also be provided that the particles of the plurality of deformable, and in particular expandable, particles are present in at least two different particle sizes. In one alternative embodiment, it may also be provided in particular that the particles of the plurality of deformable, and in particular expandable, particles are present in two different particle sizes. In one alternative embodiment, it may also be provided in particular that the particles of the plurality of deformable, and in particular expandable, particles are present in three different particle sizes. In one alternative embodiment, it may also be provided in particular that the particles of the plurality of deformable, and in particular expandable, particles are present in four different particle sizes. In one alternative embodiment, it may also be provided in particular that the particles of the plurality of deformable, and in particular expandable, particles are present in five different particle sizes.

In one alternative embodiment, it may also be provided in particular that the particles of the plurality of deformable, and in particular expandable, particles of the granulated material mixture are present in one to ten, and in particular in one to five, or in two to ten, and in particular in two to five, different particles sizes.

In one alternative embodiment, it may also be provided in particular that the particles of the plurality of deformable, and in particular expandable, particles of the granulated material mixture are present in a number of different particle sizes.

According to the invention, the at least one deformable particle can preferably expand, shrink and/or vary in shape in another manner, for example by changing the surface contour.

According to the invention, the at least one deformable particle is preferably expandable and/or shrinkable. According to the invention, the at least one deformable particle is preferably expandable or shrinkable.

According to the invention, the at least one deformable particle is preferably expandable.

According to the invention, the deformable particles are preferably expandable particles.

According to the invention, the deformable particles can preferably expand in a predefined and controlled manner as a function of the action of a force. According to the invention, the deformable particles can preferably deform in a predefined and controlled manner as a function of the action of a force.

According to the invention, the at least one deformable particle is preferably shrinkable.

According to the invention, the at least one deformable particle is preferably deformed by way of a change in volume of the particle.

In the context of the present invention, the “volume” of the deformable particles, and notably of the swelling agent, shall be understood to mean the volume that is bounded by the outer surfaces of the particles or of the swelling agent. The deformable particles, in a preferred form, are present in a starting volume, preferably the original starting volume, which can change into another volume as a result of contact with a liquid, and in particular also as a result of absorption of liquid. A change in volume denotes a change in the starting volume, in particular a significant change in the starting volume, and preferably an increase in the starting volume. For example, the change may be a change in the starting volume of at least 1%, preferably 5%, preferably 10%, preferably 15%, preferably 20%, preferably 30%, preferably 40%, preferably 50%, preferably 60%, preferably 70%, preferably 80%, preferably 90%, and in the case of an increase preferably of at least 100%, preferably 150%, preferably 200% or preferably 300%, for example by way of expansion or deformation of the particles.

In one embodiment according to the invention, the deformation, and in particular the expansion, of a deformable particle takes place in all three directions in space. In one alternative embodiment according to the invention, the deformation, and in particular the expansion, takes place directed in one or two directions in space. In one alternative embodiment according to the invention, the deformation, and in particular the expansion, takes place directed in one direction in space. A directed deformation, and in particular expansion, can be achieved, for example, by way of a casing, in particular a casing having a bellows. However, the directed expansion can also be achieved, for example, by way of a covering that has differing thicknesses in various locations and therefore dissolves at differing rates. A deformation, and in particular an expansion, then takes place in regions of the swelling agent where the covering is already dissolved, and not in locations where the covering has not yet dissolved.

According to the invention, the change in volume of the deformable particles is preferably triggered by the particles, for example by the swelling agent, making contact with and absorbing liquid, preferably a liquid comprising biomolecules and/or cells, particularly preferably blood. According to the invention, the liquid is preferably water. According to the invention, the liquid is preferably a body fluid. According to the invention, the liquid is preferably an interstitial fluid. According to the invention, the liquid is preferably blood. According to the invention, the absorbed liquid preferably comprises no solid constituents of more than 150 kDa, particularly preferably more than 100 kDa, and in particular more than 50 kDa.

According to the invention, the change in volume of the deformable particles is preferably an increase in volume.

By providing the granulated material mixture according to the invention, it is possible to introduce this mixture into a bone defect, for example surgically. After introduction into the bone defect, according to the invention the volume of the deformable particles changes, for example increases or decreases, but in particular expands, due to contact with a liquid and an associated migration of liquid, in particular an absorption of liquid. The change in volume of the deformable particles causes a change in the shape and/or size of the deformable particles, particularly preferably an increase in the surface and thus in the enclosed volume. This causes osteogenic cells or cell aggregations, which have migrated into the bone defect after introduction of the granulated material mixture into the bone defect and are adhering to the particles, in particular to the deformed particles and the non-deformed particles, to be exposed slowly and in a defined manner to stress, which is to say a biomechanical stimulus, in particular insofar as the distance of these cells from the particles is such that it is effective for distraction. Three-dimensional callus distraction is achieved by the defined expansion of the deformable particles in the bone defect and the associated movement of the non-deformable particles and the related distraction of cells adhering to the particles. As a result, the precursor of a callus is suddenly created in the entire defect by way of distraction, and the callus only has to ossify. Advantageously, this stimulus will essentially reach many cells, and particularly preferably all cells, at once. According to the invention, it is possible to directly transmit biomechanical stimuli to the osteoblasts without requiring fibroblasts. The distraction can thus act on the osteoblasts with comparatively small forces. Without being bound to theory, the distraction pulses will be transmitted to the majority of osteoblasts via the non-deformable particles, in particular if the number of non-deformable particles in the granulated material mixture does not exceed that of deformable particles and/or if the non-deformable particles are larger than the non-deformable particles.

The granulated material mixture according to the invention can advantageously be used in methods, preferably in methods according to the invention, for bone regeneration, and more particularly for three-dimensional callus distraction.

According to the invention, the granulated material mixture according to the invention preferably transmits biomechanical pulses, in particular expansion stimuli or pressure stimuli, to the cells surrounding the granulated material mixture, so that these are distracted or compressed by distances of at least 0.5 μm, in particular 1 μm, more preferably 2 μm, most preferably 10 μm to preferably 100 μm, in particular particularly preferably 1000 μm, particularly preferably 1 cm, and most preferably up to 10 cm. According to the invention, the granulated material mixture according to the invention thus preferably changes the lengths and/or widths of the deformable particles by the preferred dimensions above. Due to this preferred change in dimensions of the lengths and/or widths of the deformable particles, biomechanical pulses are transmitted to the surrounding cells. For example, cells that adhere to the particles in at least two adhesion points are expanded by the change in dimensions. However, cells that surround the particles can also experience a pressure pulse as a result of the change in dimensions of the particles, for example. In addition, the pulses may be passed on through the body's own fibrin network. However, the pulses are in particular also passed on to the cells via the non-deformable particles since the non-deformable particles, which surround the deformable particles in the granulated material mixture, are likewise moved by the deformation of the deformable particles.

The deformation of the deformable particles thus causes not only the pulses to be passed on to osteoblasts, but also moves the non-deformable particles surrounding the deformable particles, so that the non-deformable particles can likewise pass pulses on to the osteoblasts as a result of the movement. The non-deformable particles can thereby enlarge the surface for adhesion of the osteoblasts and for transmission of the pulses to the osteoblasts, this surface normally being formed only by the deformable particles. This may result in a cost reduction, for example, in particular if the deformable particles are more expensive to produce than the non-deformable particles.

Surprisingly and advantageously, the pulses may also be controlled by the size, or the size mixtures, of the deformable and/or non-deformable particles, for example they can be controlled with respect to their intensity, duration and/or velocity.

Surprisingly and advantageously, the pulses may also be controlled by the mixing ratio of deformable to non-deformable particles, for example they can be controlled with respect to their intensity, duration and/or velocity.

A person skilled in the art may thus select the composition of the particles in a granulated material mixture according to the invention so that the pulses are passed on to the cells within the desired parameters, for example in terms of the distraction duration, distraction rate and/or distraction intensity.

The person skilled in the art can thus very easily influence these parameters, which is to say by simply changing the sizes and/or the mixing ratio of the particles in the granulated material mixture. A granulated material mixture according to the invention is additionally easy and cost-effective to produce. For example, conventional non-deformable particles from the prior art may be mixed with the deformable particles without major expenditure.

According to the invention, the biomechanical pulses are preferably transmitted at a distraction rate of no more than 1 mm/day. According to the invention, the expansion stimuli are preferably transmitted at a distraction rate of no more than 1 mm/day. According to the invention, the pressure stimuli are preferably transmitted at a distraction rate of no more than 1 mm/day.

According to the invention, the degradation kinetics of the deformable particles is preferably adapted to the time pattern of a distraction that is to be carried out using the granulated material mixture according to the invention.

According to the invention, the material of the deformable particles can preferably be expanded, shrunk and/or deformed in a predefined and controlled manner as a function of the action of an external force. The material may have plastic or elastic properties. These properties of the material allow for the capacity of the deformable particles that is provided according to the invention to reversibly or irreversibly change the volumes thereof in a predefined and controlled manner.

According to the invention, the starting volume of deformable particles preferably changes at a predetermined rate. According to the invention, the maximum rate at which the starting volume of the deformable particles can preferably change is preferably such that the cells adhering to the particles, which is to say to the deformable particles and/or to the non-deformable particles, and/or the cells surrounding the particles are distracted and/or compressed no more than 1.5 mm/day, particularly preferably 1.2 mm/day, in particular 1 mm/day, and most preferably 0.9 mm/day.

In one preferred embodiment, the volume of the deformable particles may change in a predefined and controlled manner at a rate at which an expansion or shrinkage by a volume of 1000 μm³ to 216,000 μm³ occurs at no more than 0.6 mm per day, in at least one space coordinate, particularly preferably at no more than 0.577 mm per day, in particular no more than 0.55 mm per day, and most preferably no more than 0.5 mm per day. In one preferred embodiment, the volume may change in a predefined and controlled manner at a rate at which an expansion or shrinkage by a volume of 1000 μm³ to 216,000 μm³ in at least one space coordinate occurs at a minimum of 0.01 mm per day, particularly preferably at a minimum of 0.1 mm per day, in particular at a minimum of 0.2 mm per day, and most preferably at a minimum of 0.5 mm per day.

In one preferred embodiment, the volume of the deformable particles may change in a predefined and controlled manner at a rate at which an expansion or shrinkage of a section, measuring between 10 μm and 60 μm in length, of the space diagonal of the volume of the swelling agent occurs at no more than 0.6 mm per day, particularly preferably at no more than 0.577 mm per day, in particular no more than 0.55 mm per day, and most preferably no more than 0.5 mm per day. In one preferred embodiment, the volume may change in a predefined and controlled manner at a rate at which an expansion or shrinkage of a section, measuring between 10 μm and 60 μm in length, of the space diagonal of the volume of the swelling agent occurs at a minimum of 0.01 mm per day, particularly preferably at a minimum of 0.1 mm per day, in particular at a minimum of 0.2 mm per day, and most preferably at a minimum of 0.5 mm per day.

According to the invention, the deformable particles are preferably designed so that the starting volume of the deformable particles can be changed continuously. According to the invention, the deformable particles are preferably designed so that the starting volume of the deformable particles can be changed discontinuously.

In the context of the present invention, “in a predefined and controlled manner” shall be understood to mean a change in the starting volume, in particular an expansion or shrinkage, that takes place over a predetermined distance and/or a predetermined volume and the speed of which, which is to say the expansion speed, shrinkage speed or volume change speed, is likewise predetermined, which is to say deliberately selected. According to the invention, a change in the volume may also be only a change in the form of the volume. According to the invention, the time at which the expansion, shrinkage or change in volume starts can also preferably be predetermined, which is to say deliberately selected.

In the context of the present invention, an “expansion” shall be understood to mean an enlargement of the deformable particles along at least one spatial axis. According to the invention, the enlargement preferably takes place along one spatial axis. According to the invention, the enlargement preferably takes place along two spatial axes. According to the invention, the enlargement preferably takes place along all three spatial axes.

In the context of the present invention, “shrinkage” shall be understood to mean a diminution of the deformable particles along at least one spatial axis, preferably along one spatial axis, two spatial axes or all three spatial axes.

According to the invention, at least one deformable particle is mixed with at least one non deformable particle.

In one embodiment, it may be provided in particular that the at least one non-deformable particle comprises a bone substitute material.

In one embodiment, it may be provided in particular that the at least one non-expandable particle comprises a bone substitute material.

In one embodiment, it may be provided in particular that the bone substitute material is an organic or an inorganic bone substitute material.

In one embodiment, it may be provided in particular that the bone substitute material is allogeneic or autogenous bone.

In one embodiment, it may be provided in particular that the at least one non-deformable particle comprises hydroxylapatite and/or tricalcium phosphate.

In one embodiment, it may be provided in particular that the at least one non-expandable particle comprises hydroxylapatite and/or tricalcium phosphate.

In one embodiment, it may be provided in particular that the at least one non-deformable particle comprises hydroxylapatite. In one embodiment, it may be provided in particular that the at least one non-deformable particle comprises tricalcium phosphate. In one embodiment, it may be provided in particular that the at least one non-deformable particle consists of hydroxylapatite. In one embodiment, it may also be provided in particular that the at least one non-deformable particle consists of tricalcium phosphate.

In one embodiment, it may be provided in particular that the at least one non-expandable particle comprises hydroxylapatite. In one embodiment, it may be provided in particular that the at least one non-expandable particle comprises tricalcium phosphate. In one embodiment, it may be provided in particular that the at least one non-expandable particle consists of hydroxylapatite. In one embodiment, it may also be provided in particular that the at least one non-expandable particle consists of tricalcium phosphate.

In one embodiment, it may be provided in particular that the plurality of non-deformable, and in particular non-expandable, particles are made of the same material or different materials.

In one alternative embodiment according to the invention, the non-deformable particles are porous. In one alternative embodiment according to the invention, the non-deformable particles are not porous.

According to the invention, the at least one non-deformable particle is preferably produced in vitro.

In one alternative embodiment according to the invention, the non-deformable particles are particles known on the market, such as Bio-Oss® from Geistlich Pharma AG, BONIT Matrix® from DOT GmbH, or cyclOS® and Ceros® from Mathys AG.

According to the invention, the at least one non-deformable particle preferably has a particle size of 0.1 μm to 50 mm.

According to the invention, the at least one non-expandable particle preferably has a particle size of 0.1 μm to 50 mm.

In one embodiment according to the invention, the at least one non-deformable particle has a particle size of 1 μm to 50 mm.

In one embodiment according to the invention, the at least one non-expandable particle has a particle size of 1 μm to 50 mm.

In one embodiment according to the invention, the at least one non-deformable particle has a particle size of 0.01 mm to 10 mm.

In one embodiment according to the invention, the at east one non-expandable particle has a particle size of 0.01 mm to 10 mm.

In one embodiment according to the invention, the at least one non-deformable particle has a particle size of 0.1 mm to 10 mm.

In one embodiment according to the invention, the at least one non-expandable particle has a particle size of 0.1 mm to 10 mm.

In one embodiment according to the invention, the particle size is 0.2 mm to 5 mm. In one embodiment according to the invention, the particle size is 0.5 mm to 5 mm. In one embodiment according to the invention, the particle size is 0.6 mm to 5 mm. In one embodiment according to the invention, the particle size is 0.5 mm to 4 mm.

In one embodiment according to the invention, the particle size is at least 0.1 mm. In one embodiment according to the invention, the particle size is at least 0.2 mm. In one embodiment according to the invention, the particle size is at least 0.3 mm. In one embodiment according to the invention, the particle size is at least 0.4 mm. In one embodiment according to the invention, the particle size is at least 0.5 mm. In one embodiment according to the invention, the particle size is at least 0.6 mm.

In one embodiment according to the invention, the particle size is no more than 10 mm. In one embodiment according to the invention, the particle size is no more than 5 mm. In one embodiment according to the invention, the particle size is no more than 4 mm. In one embodiment according to the invention, the particle size is no more than 2 mm. In one embodiment according to the invention, the particle size is no more than 1 mm.

In one alternative embodiment, it may be provided in particular that the particles of the plurality of non-deformable, and in particular non-expandable, particles are present in a single particle size.

However, in one alternative embodiment, it may also be provided that the particles of the plurality of non-deformable, and in particular non-expandable, particles are present in at least two different particle sizes. In one alternative embodiment, it may also be provided in particular that the particles of the plurality of non-deformable, and in particular non-expandable, particles are present in two different particle sizes. In one alternative embodiment, it may also be provided in particular that the particles of the plurality of non-deformable, and in particular non-expandable, particles are present in three different particle sizes. In one alternative embodiment, it may also be provided in particular that the particles of the plurality of non-deformable, and in particular non-expandable, particles are present in four different particle sizes. In one alternative embodiment, it may also be provided in particular that the particles of the plurality of non-deformable, and in particular non-expandable, particles are present in five different particle sizes.

In one alternative embodiment, it may also be provided in particular that the particles of the plurality of non-deformable, and in particular non-expandable, particles of the granulated material mixture are present in one to ten, and in particular in one to five, or in two to ten, and in particular in two to five, different particles sizes.

In one alternative embodiment, it may also be provided in particular that the particles of the plurality of non-deformable, and in particular non-expandable, particles of the granulated material mixture are present in a number of different particle sizes.

In one alternative embodiment according to the invention, the non-deformable particles in the granulated material mixture are larger than the deformable particles. In one alternative embodiment according to the invention, the non-deformable particles in the granulated material mixture are up to 10 times larger, and more particularly up to 100 times larger, than the deformable particles.

According to the invention, the at least one non-deformable particle can preferably not expand, not shrink and/or not vary in shape in any other manner such as by changing the surface contour. According to the invention, the at least one non-deformable particle is preferably non-expandable and/or non-shrinkable. According to the invention, the at least one non-deformable particle is preferably non-expandable and non-shrinkable.

According to the invention, the at least one non-deformable particle is preferably non-expandable.

According to the invention, the non-deformable particles are preferably non-expandable and non-shrinkable particles.

According to the invention, the non-deformable particles are preferably rigid particles. According to the invention, preferably no change in volume takes place in the non-deformable particles, such as upon contact of the particles with a liquid.

The granulated material mixture is preferably not embedded in a non-expandable polymeric matrix, in particular in a matrix, especially prior to being used.

In a preferred embodiment, the granulated material mixture comprises cells, for example stem cells, in addition to the deformable and non-deformable particles.

In one preferred embodiment according to the invention, the granulated material mixture comprises growth factors, in addition to the deformable and non-deformable particles. The growth factors may be bound to the particles, for example to the deformable particles and/or the non-deformable particles. However, the growth factors may also not be bound to the particles.

In one preferred embodiment, the at least one non-deformable particle comprises mineral constituents of bone. In one preferred embodiment according to the invention, the at least one non-deformable particle consists of mineral constituents of bone. For example, the constituents of bone may be of bovine origin, such as Bio-Oss®. In one preferred embodiment according to the invention, the at least one non-deformable particle consists of constituents of marine algae origin, such as Frios Algipore®, or comprises the same.

In one preferred embodiment, the at least one non-deformable particle comprises plant and/or animal hydroxylapatite. In one preferred embodiment according to the invention, the at least one non-deformable particle consists of plant and/or animal hydroxylapatite.

In one preferred embodiment, the at least one non-deformable particle comprises a synthetic bone substitute material, for example a resorbable, pure-phase β-tricalcium phosphate matrix, preferably having open, interconnecting porosity, such as Cerasorb®. In one preferred embodiment according to the invention, the at least one non-deformable particle consists of such a synthetic bone substitute material.

In one preferred embodiment, the at least one non-deformable particle has a diameter of at least 0.25 mm to no more than 1 mm. In one preferred embodiment according to the invention, the at least one non-deformable particle has a diameter of at least 1 mm to no more than 2 mm. In one preferred embodiment according to the invention, the at least one non-deformable particle has a diameter of at least 0.3 mm to no more than 5 mm. In one preferred embodiment according to the invention, the at least one non-deformable particle has a diameter of at least 50 μm to no more than 150 μm, and more particularly no more than 250 μm. In one preferred embodiment according to the invention, the at least one non-deformable particle has a diameter of at least 50 μm to no more than 2 mm.

In one preferred embodiment, the at least one deformable, and in particular expandable, particle is a core-shell particle. The particle thus preferably comprises a core and a casing. The at least one expandable particle, and more particularly the core of the particle, preferably comprises a hydrogel as the swelling agent. The hydrogel is preferably enclosed by a degradable, and more particularly biodegradable, covering. The hydrogel is preferably degradable, and more particularly biodegradable.

In one preferred embodiment, the at least one deformable, and in particular expandable, particle has a diameter of approximately 10 μm to approximately 1 mm. In one preferred embodiment, the at least one deformable, and in particular expandable, particle has a diameter of no more than 1 mm. In one preferred embodiment, the at least one deformable, and in particular expandable, particle has a diameter of 250 μm to 1 mm. In one preferred embodiment, the at least one deformable, and in particular expandable, particle has a diameter of 300 μm to 500 μm. In one preferred embodiment, the at least one deformable, and in particular expandable, particle has a diameter of 0.5 mm to 1 mm. In one preferred embodiment, the at least one deformable, and in particular expandable, particle has a diameter of 100 μm to 500 μm. In one preferred embodiment, the at least one deformable, and in particular expandable, particle has a diameter of at least 10 μm, in particular at least 50 μm, in particular at least 100 μm, in particular at least 200 μm, in particular at least 300 μm, in particular at east 400 μm, and in particular at least 500 μm.

The hydrogel preferably consists of carboxymethylcellulose, or comprises the same. The hydrogel preferably consists of chitosan and carboxymethylcellulose, or comprises the same. The hydrogel preferably consists of a mixture of chitosan and carboxymethylcellulose. The hydrogel is preferably antimicrobial.

The hydrogel preferably consists of hyaluronic acid, or comprises the same. The hydrogel preferably consists of chitosan and hyaluronic acid, or comprises the same. The hydrogel preferably consists of a mixture of chitosan and hyaluronic acid.

The hydrogel preferably consists of alginate, or comprises the same. The hydrogel preferably consists of chitosan and alginate, or comprises the same. The hydrogel preferably consists of a mixture of chitosan and alginate. The hydrogel preferably consists of polyethylene glycol and alginate, or comprises the same. The hydrogel preferably consists of a mixture of polyethylene glycol and alginate. The hydrogel preferably consists of polyethylene quinine and alginate, or comprises the same. The hydrogel preferably consists of a mixture of polyethylene quinine and alginate.

Chitosan is preferably used to cross-link the hydrogel.

The hydrogel can preferably increase in size, which is to say expand, by up to 10 times, and particularly preferably by up to 25 times, the starting volume thereof. The hydrogel can preferably expand continuously.

The hydrogel preferably has a spherical shape. Surfactants are preferably used to form the spherical shape.

In one preferred embodiment, the at least one deformable, and in particular expandable, particle comprises a covering or a casing. The covering is preferably degradable, and more particularly biodegradable. The covering is preferably degraded by way of hydrolysis.

The covering is preferably degraded more quickly than the hydrogel. The covering is preferably degraded within 2 weeks, in particular within 10 days, and preferably within one week, in particular by way of hydrolysis. The hydrogel can expand after the casing has partially or completely degraded. The casing thus prevents the hydrogel from expanding due to the force acting on the hydrogel. As an alternative or in addition, the casing may prevent the hydrogel from expanding by shielding the hydrogel from liquids, for example water or blood.

The casing is preferably water-permeable. The casing is preferably not water-permeable.

A hydrogel particle is preferably encapsulated by a casing. However, a plurality of hydrogel particles may also be collectively encapsulated by a single casing.

The casing preferably comprises polylactic acid. The casing preferably comprises polyglycolic acid. The casing preferably comprises polylactic acid and polyglycolic acid. The casing preferably consists of polylactic acid and polyglycolic acid. The casing preferably comprises a copolymer made of polylactic acid and polyglycolic acid. The casing preferably consists of a copolymer made of polylactic acid and polyglycolic acid. The copolymer preferably comprises 1% by weight to 99% by weight polylactic acid. The copolymer preferably comprises 2% by weight to 98% by weight polylactic acid and 98% by weight to 2% by weight polyglycolic acid. The copolymer preferably comprises 10% by weight to 80% by weight polylactic acid. The copolymer preferably comprises 25% by weight to 75% by weight polylactic acid and 75% by weight to 25% by weight polyglycolic acid.

The duration over which the covering prevents the hydrogel from expanding, in particular by the action of a force, is preferably determined by the thickness of the covering. The diameter of a hydrogel particle preferably corresponds to several times the thickness of the covering. The wall thickness of the covering is thus preferably several times smaller than the diameter of the covering.

The particles, and more particularly the expandable particles and/or the non-deformable particles, can preferably be sterilized.

In one preferred embodiment, the covering comprises growth factors. In one preferred embodiment, the covering comprises bone particles or bone substitute material particles.

In one preferred embodiment, the covering has pores. The covering may be designed as a framework, for example. In one preferred embodiment, the covering has no pores. For example, the covering may be formed as a casing having no pores, for example as a layer or film on the hydrogel.

The present invention also relates to a method for producing a granulated material mixture according to the invention, wherein at least one deformable, and in particular expandable, particle, and more particularly a plurality of deformable, and in particular expandable, particles, and at least one non-deformable, and in particular non-expandable, particle, and more particularly a plurality of non-deformable, and in particular non-expandable, particles, are mixed.

The present invention also relates to a method for regenerating a bone, wherein at least one granulated material mixture according to the invention is introduced into a defect region of a bone.

The present invention also relates to medical procedures in which a granulated material mixture according to the invention is used.

The invention thus also relates to the first medical indication for a granulated material mixture made of deformable, and in particular expandable, particles and non-deformable, and in particular non-expandable, particles, in particular of a granulated material mixture according to the invention.

In one embodiment according to the invention, the granulated material mixture is introduced into a defect region of a bone in such a way that the at least one deformable, and in particular expandable, particle comes in contact with a liquid.

In one embodiment according to the invention, the bone defect is refreshed before the granulated material mixture is introduced.

Accordingly, within the framework of the method for bone regeneration according to the invention, in one preferred embodiment a granulated material mixture made of deformable and non-deformable particles, and more particularly a granulated material mixture according to the invention, is introduced into a defect region of a bone. The granulated material mixture is enclosed by a blood clot in this defect region, which is to say the surfaces of the particles make contact with the autologous cells present in the blood clot. After the granulated material mixture has been introduced into the defect region of a bone, a change in volume, which is to say in particular a decrease or increase in volume, of the deformable particles is triggered by a liquid. This results in an expansion and/or change in shape, and thus in the desired biomechanical stimulation, of the osteogenic cells attached to the deformable and the non-deformable particles, and consequently results in distraction and thus bone regeneration. According to the invention, the action of the force preferably takes place within the body, and more particularly within the bone defect.

According to the invention, the change in volume of the deformable particles may preferably be of various orders of magnitude. The change is preferably approximately 10% of the longitudinal extension of the cells, or cell groups, adhering to the deformable particles.

According to the invention, the change in the expansion distance is preferably at least 0.5 μm, particularly preferably at least 1 μm, more preferably at least 10 μm, still more preferably at least 100 μm, very preferably at least 1000 μm, very particularly preferably at least 10 mm, and most preferably at least 100 mm.

According to the invention, the change in the expansion distance is preferably no more than 100 mm, particularly preferably no more than 10 mm, more preferably no more than 1000 μm, still more preferably at least 100 μm, very preferably no more than 10 μm, very particularly preferably no more than 1 μm, and most preferably no more than 0.5 μm.

According to the invention, the distraction distance is preferably 5 mm to 10 mm.

According to the invention, the distraction force of the deformable particles preferably must be greater than the shrinkage force of the fibrin scaffold or blood clot.

According to the invention, distraction as a result of the deformation, expansion or shrinkage of the deformable particles preferably begins one day after the granulated material mixture has been introduced into the bone defect. According to the invention, distraction as a result of the deformation, expansion or shrinkage of the deformable particles preferably begins one week after the granulated material mixture has been introduced into the bone defect. The start of distraction may be dictated by a covering of the deformable particles, and more particularly by the thickness of the covering.

In one embodiment according to the invention, the distraction takes place over a period of several days or weeks. In one embodiment according to the invention, the distraction takes place over a period of several days. In one embodiment according to the invention, the distraction takes place over a period of several weeks.

In one embodiment according to the invention, the distraction takes place over a period of at least 1 day, in particular at least 2 days, and no more than 300 days, in particular no more than 100 days.

In one embodiment according to the invention, the distraction takes place over a period of at least 1 day. In one embodiment according to the invention, the distraction takes place over a period of at least 2 days. In one embodiment according to the invention, the distraction takes place over a period of at least 5 days. In one embodiment according to the invention, the distraction takes place over a period of at least 10 days.

In one embodiment according to the invention, the distraction takes place over a period of no more than 300 days. In one embodiment according to the invention, the distraction takes place over a period of no more than 100 days. In one embodiment according to the invention, the distraction takes place over a period of no more than 50 days.

In one embodiment according to the invention, the distraction takes place over a period of several days, in particular over a period of 5 to 20 days, particularly preferably over a period of approximately 10 days, and more particularly of 10 days.

According to the invention, the minimum rate of change in the volume is preferably such that cells adhering to the particles are distracted at least 1 μm/day. According to the invention, the rate of change in the volume is preferably such that cells adhering to the particles are distracted between 0.5 mm/day and 1 mm/day. According to the invention, the maximum rate of change in the volume is preferably such that cells adhering to the particles are distracted, or osteogenic, callus-producing tissue is distracted, no more than 1 mm/day. A distraction rate faster than 1 mm/day results in the differentiation of connective tissue instead of bone. As a result of the change in volume, the deformable particles transmit biomechanical stimuli to the cells that are present in the blood clot and to cells adhering to the deformable and non-deformable particles, these stimuli triggering the body's own regenerative forces, whereby new autologous bone material forms. This material does not differ from the original bone material surrounding the defect. The change in volume of the deformable particles results in biomechanical stimuli transmission across the entire area that is taken up by the granulated material mixture, whereby a biomechanical stimulus is transmitted to considerably more cells than with distraction osteogenesis from the prior art. According to the invention, the biomechanical stimulus is preferably transmitted both from the deformable particles directly to osteoblasts and from the non-deformable particles to the osteoblasts.

In one embodiment according to the invention, the distraction takes place in all three directions in space. In one alternative embodiment according to the invention, the distraction takes place directed in one or two directions in space. In one alternative embodiment according to the invention, the distraction takes place directed in one direction in space. Without being bound to theory, it may be advantageous in some situations to have the distraction take place directed in one direction in space, so that the distraction follows a possible orientation of the fibers.

With a distraction according to the invention, the biomechanical stimuli can preferably be transmitted, according to the invention, not only directly to osteoblasts that adhere to the particles, but also indirectly by way of fibroblasts. According to the invention, fibroblasts adhering to the particles preferably further transmit the distraction stimulus to osteoblasts in a metered manner. Without being bound to theory, fibroblasts in what is referred to as the “null zone” also turn into osteoblasts after the distraction is completed and likewise form bone. When the distraction speed decreases, the number of fibroblasts preceding the osteoblasts changes.

In contrast, distraction osteogenesis from the prior art transmits biomechanical stimuli via a two-dimensional interface made of bone or another material only to those cells that are in direct contact with this two-dimensional interface.

Without being bound to theory, the method according to the invention can achieve both cell distraction and tissue distraction.

In the context of the present invention, cell distraction shall be understood to mean distraction of individual cells, in particular osteoblasts. These individual cells attach to the deformable or non-deformable particles and, either directly or indirectly, experience distraction pulses due to the deformation of the deformable particles. In one embodiment according to the invention, a distraction pulse that a cell, in particular an osteoblast, experiences is 1 μm to 10 μm. In one embodiment according to the invention, the distraction distance that a cell, in particular an osteoblast, is pulled is 1 μm to 200 μm. In one embodiment according to the invention, the distraction distance that a cell, in particular an osteoblast, is pulled is at least 1 μm and no more than 10 μm. In one embodiment according to the invention, the distraction distance that a cell, in particular an osteoblast, is pulled is at least 10 μm and no more than 200 μm.

In one embodiment according to the invention, the rate at which a cell, in particular an osteoblast, is pulled is at least 1 μm/day.

In the context of the present invention, tissue distraction shall be understood to mean the distraction of a tissue, for example of a bone tissue, and in particular of a callus. The tissue is composed of a plurality of cells, in particular also osteoblasts. The tissue, in particular a callus, attaches to the deformable or non-deformable particles and, either directly or indirectly, experiences distraction pulses due to the deformation of the deformable particles. In one embodiment according to the invention, the distraction pulse that a tissue, in particular a callus, experiences is 1 μm to 1000 μm. In one embodiment according to the invention, the distraction distance that a tissue, in particular a callus, is pulled is 10 μm to 30 cm. In one embodiment according to the invention, the distraction distance that a tissue, in particular a callus, is pulled is 10 μm to 3 cm. In one embodiment according to the invention, the distraction distance that a tissue, in particular a callus, is pulled is 10 μm to 10 mm. In one embodiment according to the invention, the distraction distance that a tissue, in particular a callus, is pulled is at least 0.2 mm to no more than 5 mm.

In one embodiment according to the invention, the distraction distance that a tissue, in particular a callus, is pulled is at least 10 μm. In one embodiment according to the invention, the distraction distance that a tissue, in particular a callus, is pulled is at least 100 μm. In one embodiment according to the invention, the distraction distance that a tissue, in particular a callus, is pulled is at least 1 mm. In one embodiment according to the invention, the distraction distance that a tissue, in particular a callus, is pulled is no more than 30 cm. In one embodiment according to the invention, the distraction distance that a tissue, in particular a callus, is pulled is no more than 10 cm. In one embodiment according to the invention, the distraction distance that a tissue, in particular a callus, is pulled is no more than 3 cm. In one embodiment according to the invention, the distraction distance that a tissue, in particular a callus, is pulled is no more than 1 cm. In one embodiment according to the invention, the distraction distance that a tissue, in particular a callus, is pulled is no more than 0.5 cm.

In one embodiment according to the invention, the rate at which a tissue, in particular a callus, is pulled is at least 10 μm/day. In one embodiment according to the invention, the rate at which a tissue, in particular a callus, is pulled is at least 0.1 mm/day. In one embodiment according to the invention, the rate at which a tissue, in particular a callus, is pulled is at least 0.25 mm/day. In one embodiment according to the invention, the rate at which a tissue, in particular a callus, is pulled is no more than 2 mm/day. In one embodiment according to the invention, the rate at which a tissue, in particular a callus, is pulled is approximately 1 mm/day.

In one embodiment according to the invention, the rate at which a tissue, in particular a callus, is pulled is at least 0.25 mm/day and no more than 2 mm/day. In one embodiment according to the invention, the rate at which a tissue, in particular a callus, is pulled is at least 0.5 mm/day and no more than 2 mm/day. In one embodiment according to the invention, the rate at which a tissue, in particular a callus, is pulled is at least 0.5 mm/day and no more than 1.5 mm/day.

The invention thus provides a method in which a granulated material mixture made of deformable and non-deformable particles is introduced into a bone defect and the deformable particles change in volume and/or shape in the bone defect. As a result of the change in volume and/or shape, biomechanical stimuli are transmitted to cells, in particular osteoblasts, which are located on the outer surface of the deformable and non-deformable particles, whereby the cells are stimulated so as to form bone. The particles thus transmit biomechanical stimuli so as to utilize the body's own regenerative forces.

The method according to the invention is therefore a three-dimensional distraction. In the context of the present invention, “three-dimensional distraction” shall be understood to mean distractive bone regeneration in which not only are biomechanical stimuli transmitted to a bone fragment at the interface, which is to say two-dimensionally, but stimuli are also transmitted across a particular volume, which is to say three-dimensionally.

According to the invention, it may preferably be provided that the distraction takes place along a spatial axis. This can be done, for example, by using an alternative embodiment of the deformable particles, in which the lengths of the particles are changed, for example by way of a bellows.

The method according to the invention utilizes the body's own wound healing mechanisms as a bioreactor. Osteogenesis thus occurs under natural conditions, so that the necessary aspects such as growth factors, hormones and cell composition are implicitly taken into consideration. The method according to the invention thus overcomes not only problems that may arise due to the highly complex control in bone regeneration, but also the problems of slow and complicated bone regeneration by distraction methods from the prior art.

According to the invention, the bone defect is preferably refreshed before the device according to the invention is introduced. According to the invention, a bone defect is surgically refreshed with the method according to the invention, and in particular bleeding is induced, before the device according to the invention is introduced into this defect. A blood clot forms in the defect as a result of the surgical refreshment and the induced bleeding.

After the bone defect has been surgically refreshed, according to the invention a granulated material mixture according to the invention is preferably introduced into the bone defect. The particles are surrounded, in particularly completely surrounded, by the blood clot that has formed. To this end, according to the invention the deformable particles, and for example also the swelling agent forming the particles, preferably come in contact with a liquid, such as the blood of the blood clot.

According to the invention, the granulated material mixture is preferably introduced into a defect region of a bone in such a way that the swelling agent of the deformable particles comes in contact with a liquid.

According to the invention, the deformable particles thus preferably change in volume after a defined point in time. According to the invention, the deformable particles preferably change in volumes after one day. According to the invention, the deformable particles preferably change in volumes after one week. Without being bound to theory, the blood clot will not shrink, but will increase in keeping with the volume increase of the deformable particles. The cells activated by the granulated material mixture may be converted into proliferating osteoblasts, which produce extracellular matrix, and a callus may be formed, which subsequently ossifies. If the deformable particles are preferably biodegradable in accordance with the invention, these will subsequently be resorbed and/or metabolized. The bone defect can thus fill with bone tissue, which according to the invention has preferably been produced by the described biomechanical stimuli of the granulated material mixture. According to the invention, aside from the granulated material mixture, growth factors and other substances may preferably be dispensed with. According to the invention, the newly formed bone material preferably differs only little, if at all, from the original bone surrounding it, either histologically or in terms of the biological or medical value thereof.

According to the invention, the resorption time for the deformable particles is preferably approximately 1 to 2 years, particularly preferably approximately 1.5 years, and in particular 1.5 years.

Because, according to the invention, the deformable particles are preferably biodegradable, the space that results from the degradation of the device may be used for the extracellular matrix. According to the invention, the degradation of the deformable particles can preferably be adjusted so that, after just a few weeks, the particles degrade after the biomechanical stimuli have been emitted and the resulting space has been taken up by the extracellular matrix.

In one alternative according to the invention, deformable particles, the casings of which have cell-adhesive properties, may be used within the framework of the method according to the invention. It is particularly preferred if the surface of the casing has cell-adhesive properties. The surface of the casing plays a role in the attachment of cells from the blood clot. Preferred adhesion of the cells to the casing according to the invention may be influenced by way of the surface chemistry and surface physics as well as by way of the surface topography of the casing. According to the invention, the surface of the casing is preferably hydrophilic. For the ingrowing cells, the interaction between the negatively charged cell membrane and the electrical properties of the surface of the casing is preferred according to the invention.

The present invention also relates to the use of a granulated material mixture according to the invention for regenerating a bone, wherein the granulated material mixture is introduced into a defect region of a bone.

The present invention also relates to the use of deformable, and in particular expandable, particles and non-deformable, and in particular non-expandable, particles for producing a granulated material mixture, in particular a granulated material mixture according to the invention, for regenerating a bone, wherein the granulated material mixture is introduced into a defect region of a bone.

The present invention also relates to a granulated material mixture, comprising at least one deformable, and in particular expandable, particle and at least one non-deformable, and in particular non-expandable, particle, in particular a granulated material mixture according to the invention, for use in the regeneration of a bone.

The invention thus also relates to the second medical indication for a granulated material mixture made of deformable, and in particular expandable, particles and non-deformable, and in particular non-expandable, particles, in particular of a granulated material mixture according to the invention, for regenerating a bone, and in particular a bone in the jaw region.

The invention also relates to the use of a granulated material mixture according to the invention for producing a kit for bone regeneration.

The invention also relates to a kit for bone regeneration, comprising a plurality of deformable, and in particular expandable, particles and a plurality of non-deformable, and in particular non-expandable, particles. In particular, the invention relates to a kit for bone regeneration, comprising a granulated material mixture according to the invention.

According to the invention, said kit preferably comprises at least one surgical instrument, particularly preferably at least one applicator, for example a syringe, and a capsule for receiving the granulated material mixture. According to the invention, the kit preferably includes instructions for use. According to the invention, the kit preferably includes packaging, particularly preferably packaging that allows sterile storage of the granulated material mixture.

According to the invention, the components of the kit are preferably associated with the granulated material mixture according to the invention.

Further devices, such as a surgical instrument, instructions for use and/or packaging may thus be associated with the granulated material mixture according to the invention.

Preferred and alternative embodiments according to the invention of the granulated material mixture according to the invention shall also be understood as preferred and alternative embodiments according to the invention of a use according to the invention, and as preferred and alternative embodiments according to the invention of a method according to the invention.

Preferred and alternative embodiments according to the invention of the method according to the invention shall also be understood as preferred and alternative embodiments according to the invention of the uses according to the invention, and as preferred and alternative embodiments according to the invention of the granulated material mixture according to the invention.

Further advantageous embodiments of the invention will be apparent from the dependent claims. The invention will be described in greater detail based on the following exemplary embodiment and the accompanying figures.

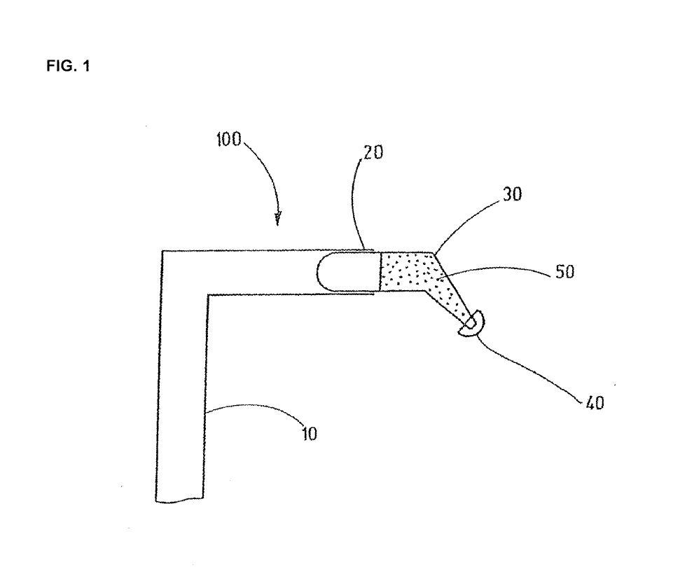

FIG. 1 is a schematic illustration of a kit, comprising a granulated material mixture in an applicator in the form of a syringe.

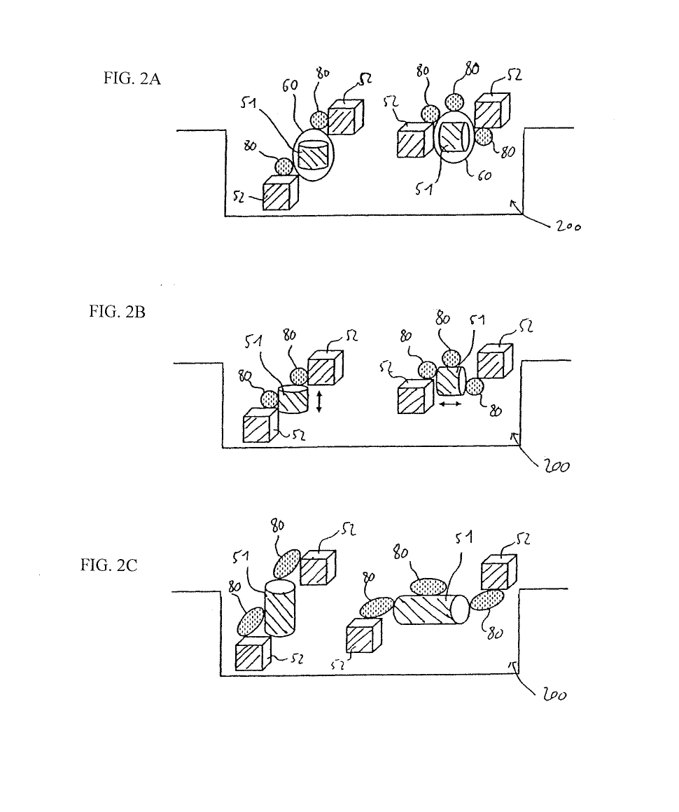

FIG. 2 is a schematic illustration of a granulated material mixture that has been introduced into a bone defect, before and after the change in volume of the expandable particles present in the mixture.

### EXAMPLE

FIG. 1 shows a kit 100, comprising an applicator tip 10 made of sterilizable material, a disposable capsule 30, for example made of plastic, being attached to the open end 20 thereof. The disposable capsule 30 is provided with a protective cap 40 toward the outside. The disposable capsule 30 holds a granulated material mixture 50 according to the invention made of deformable and non-deformable particles. The granulated material mixture is injected into a bone defect, which is not shown, for example in the jaw region, using the syringe.

The kit 100 according to the invention is used to inject the granulated material mixture 50 into a bone defect. After introduction into the bone defect, the volume of the swelling agent of the deformable particles changes due to the inventive structure and composition of the deformable particles and as a result of contact with liquid, resulting in expansion, shrinkage and/or change in shape of the deformable particles. The non-deformable particles and the bone cells, which have meanwhile attached to the deformable and non-deformable particles, are thus distracted for regeneration of the bone.

FIG. 2A shows two deformable particles 51 and several non-deformable particles 52 of a granulated material mixture 50 in a bone defect 200, more particularly immediately after the granulated material mixture 50 was introduced into the bone defect 200, for example by way of a kit 100. The deformable particles 51 are encapsulated by coverings 60. The deformable particles can be made of a swelling agent, for example. After having been introduced into the defect 200, the covering, which can be made of gelatin, for example, is biologically decomposed and degraded. FIG. 2B shows the situation after the coverings 60 have degraded. The deformable particles 51 now have direct contact with the liquid present in the bone defect, in particular blood, whereby the volumes of the deformable particles 51 increase in the longitudinal axis, as is shown schematically by the double arrows in FIG. 28. This increase in volume of the deformable particles 51 results in expanded particles 51, as is apparent in FIG. 2C. The expansion causes the surrounding non-deformable particles 52 to be pushed away, thus resulting in distraction of the cells 80 that have attached to and are adhering to the deformable and non-deformable particles 51/52.

1. A granulated material mixture suitable for regenerating a bone, comprising at least one expandable particle and at least one non-deformable particle, wherein the at least one expandable particle comprises a hydrogel as a swelling agent, and the at least one expandable particle is enclosed by a degradable covering.

2. The granulated material mixture according to claim 1, wherein the granulated material mixture comprises a plurality of expandable particles and a plurality of non-deformable particles.

3. The granulated material mixture according to claim 2, wherein the mixing ratio of the expandable particles to the non-deformable particles in the granulated material mixture, relative to the number of particles, ranges from 1:99 to 99:1.

4. The granulated material mixture according to claim 1, wherein the hydrogel comprises carboxymethylcellulose.

5. The granulated material mixture according to claim 1, wherein the hydrogel comprises chitosan/carboxymethylcellulose.

6. The granulated material mixture according to claim 1, wherein the degradable shell comprises a copolymer made of polyglycolic acid and polylactic acid.

7. The granulated material mixture according to claim 1, wherein the at least one expandable particle has a particle size from 0.0001 mm to 10 mm.

8. The granulated material mixture according to claim 1, wherein the at least one non-deformable particle comprises a bone substitute material.

9. The method for producing a granulated material mixture according to claim 2, comprising mixing a plurality of expandable particles and a plurality of non-deformable particles.

10. A method for regenerating a bone, comprising introducing at least one granulated material mixture according to claim 8 into a defect region of a bone.

11. The granulated mixture according to claim 1, wherein the degradable covering is a degradable shell.

12. The granulated material according to claim 8, wherein the bone substitute material is at least of hydroxylapatite and tricalcium phospate.

13. The granulated material mixture according to claim 1, wherein the at least one expandable particle has a particle size from 0.1 mm to 10 mm.
